(19) United States
(12) Patent Application Publication
(10) Pub. No.: US 2014/0223862 A1
(43) Pub. Date: Aug. 14, 2014

(54) Manufacturing Process For Packing Of Injectables

(71) Applicants: Fabiano Nicoletti, Mira (IT); Christer Andreasson, San Marcos, CA (US)

(72) Inventors: Fabiano Nicoletti, Mira (IT); Christer Andreasson, San Marcos, CA (US)

(73) Assignees: SAFETY SYRINGES, INC., Carlsbad, CA (US); STEVANATO GROUP INTERNATIONAL A.S., Bratislava (SK)

(21) Appl. No.: 13/809,620

(22) PCT Filed: Jul. 16, 2010
(86) PCT No.: PCT/EP2010/060332

§ 371 Date: Mar. 24, 2014

(51) Int. Cl. A61M 5/00; B65B 55/02; A61M 5/31; B65B 5/04

(52) U.S. Cl. CPC A61M 5/002; B65B 5/04; B65B 55/02; A61M 5/31; A61M 2005/3117
USPC 53/415; 53/425

(57) ABSTRACT

The manufacturing process for packing of injectables for medical use, comprises in temporal sequence the steps of:

a) forming (1) of glass primary containers,

b) forming by plastic injection (3) of complementary devices (4),

c (assembling (5) of the empty primary containers with corresponding complementary devices to form assembled devices (6),
d) washing (7) the assembled devices with water for injection,
e) nesting (8) the assembled devices in a tray (9),
f) inserting said tray housing said assembled devices into a secondary container and closing said secondary container,
g) sterilising (10) the assembled devices in the closed secondary container,
h) preparing the closed secondary container for transportation to a remote site.

The present invention relates to a manufacturing process for packaging of injectables for medical use.

Standard manufacturing processes for packing of injectables of the present art generally involve the following sequence of steps: washing, secondary packaging suitable for sterilisation, sterilisation, filling, stoppering, and final secondary packing of a primary container for the substance to be injected. Importantly, before the final secondary packing takes place, the filled primary container must be visually and/or functionally inspected to ensure that its shape and content meet the required cosmetic and functional requirements. This inspection is often compulsory in the pharmaceutical industry for regulatory purposes and is generally carried out either by way of automated systems involving cameras and corresponding software, or by operators or inspectors.

U.S. Pat. No. 6,263,641, U.S. Pat. No. 6,792,743, U.S. Pat. No. 6,189,292 and U.S. 2006/0054523 describe manufacturing processes for packing of injectables for medical use wherein an additional device, such as a safety device, is assembled onto the primary container after the filling and stoppering and outside a sterile environment. However, where the primary container is provided with a complementary device, such as where the primary container is a syringe and the complementary device is a system of preventing injuries from the bare needle tip after injection, the visual inspection must be performed both after the stoppering step and after the assembly step. This repeated checking increases the time requirement, complexity and cost of the manufacturing process.

The technical task of the present invention is therefore that of providing a manufacturing process for packing of injectables that overcomes the noted technical drawbacks of the prior art. Within the scope of this technical task is therefore that of providing a manufacturing process for packing of injectables with increased productivity.

A further object of the invention is that of providing a simple, cost-effective manufacturing process for packing of injectables while maintaining health and safety standards.

The technical task, as well as these and other objects of the invention, are achieved according to the present invention by a manufacturing process for packing of injectables for medical use, characterised in that it comprises in temporal sequence the steps of:
a) forming (1) of glass primary containers,

b) forming by plastic injection (3) of complementary devices (4),

c) assembling (5) of the empty primary containers with corresponding complementary devices to form assembled devices (6),
d) washing (7) the assembled devices with water for injection,
e) nesting (8) the assembled devices in a tray (9),
f) inserting said tray housing said assembled devices into a secondary container and closing said secondary container,
g) sterilising (10) the assembled devices in the closed secondary container,
h) preparing the closed secondary container for transportation to a remote site.

Preferably the process comprises, after the step of assembling the empty primary containers with corresponding complementary devices, a step of filling and stoppering said primary containers of said assembled devices.

The step of assembling the empty primary containers with corresponding complementary devices being before filling and stoppering negates the need for inspection after the assembly step. This in turn eliminates the time requirement and cost associated with the second check, thereby increasing productivity. Moreover, the lack of a second check simplifies the process, thereby simplifying operation and rendering the process more cost-effective while maintaining health and safety standards.

The present invention furthermore allows for the same number and type of operations to be performed inside the sterile environment as in the present art, namely transferring, positioning, filling and stoppering. The cost of adapting the processes of the prior art so as to incorporate the present invention is thus not excessive.

In addition, the manufacturing process of the present invention does not lead to any higher potential level of contamination of the sterile environment than the processes of the prior art. Such contamination may include a low particles content, low bio-burden and limited ethylene oxide residual after sterilisation.

Other characteristics of the present invention are moreover defined in the subsequent claims. Further characteristics and advantages of the present invention will be more evident from the description of a preferred, but not exclusive, embodiment of the manufacturing process for packing of injectables according to the finding, illustrated in the attached, non-limiting drawings, wherein:

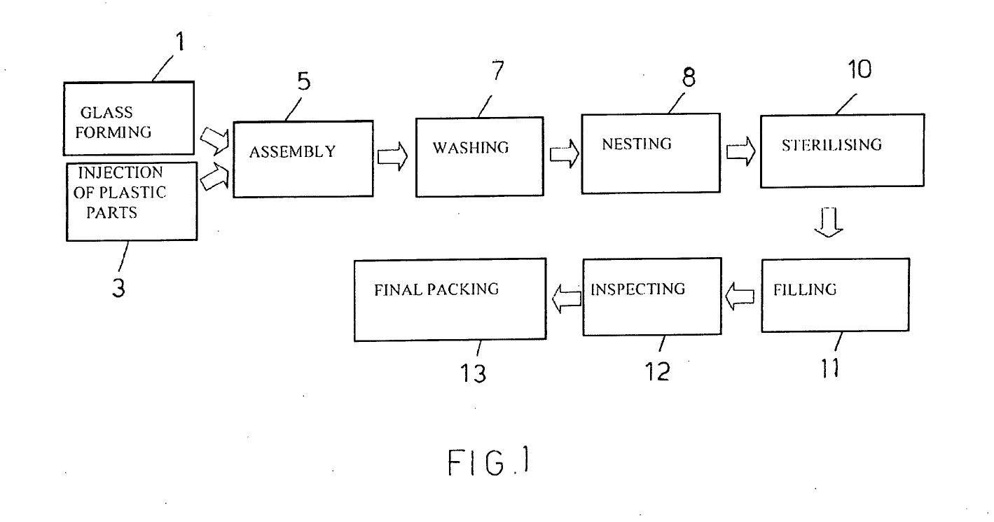

FIG. 1 shows a process flow diagram of one embodiment of the present invention.

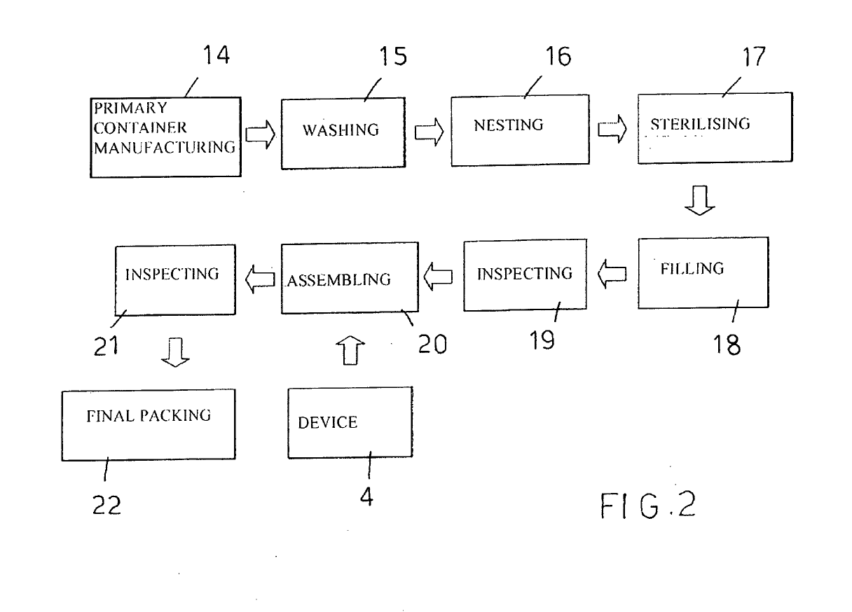

FIG. 2 shows a process flow diagram of the prior art.

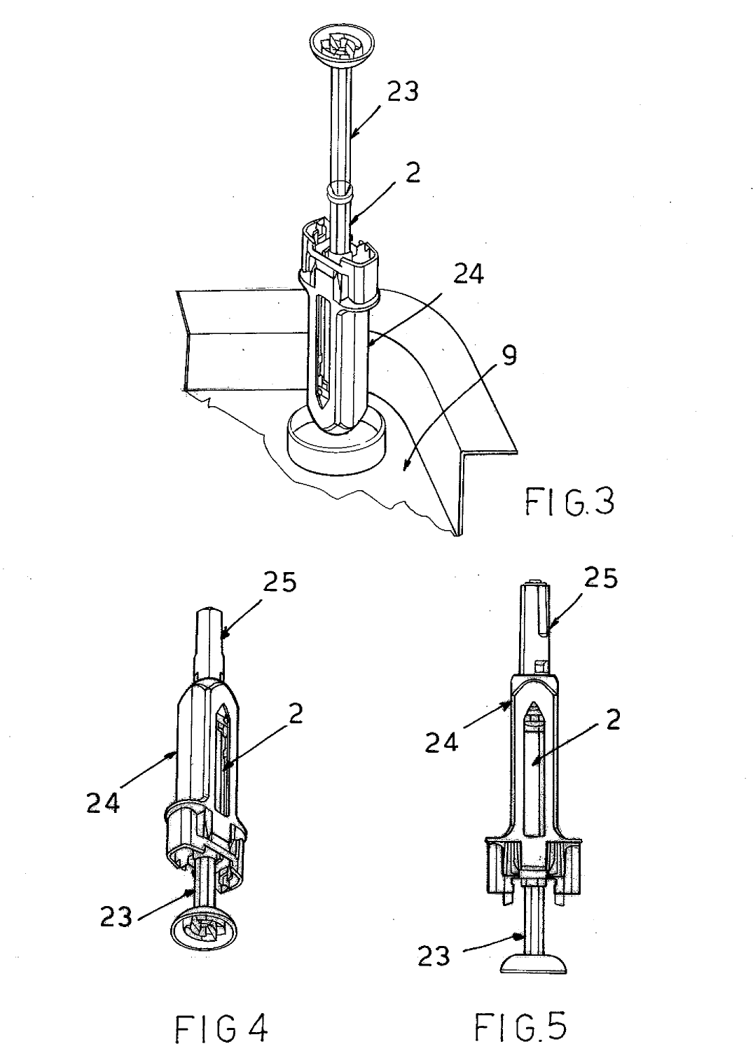

FIG. 3 shows an exploded view of an assembled device nested in a tray, filled and stoppered, according to one embodiment of the invention.

FIG. 4 shows a perspective view of one embodiment of an assembled device, filled and stoppered, according to the embodiment of the invention of FIG. 3.

FIG. 5 shows a front view of the assembled device, filled and stoppered, of FIG. 4.

Reference numbers refer to equivalent features in each of the figures.

With reference to the above figures, the invention comprises a sequence of subsequent steps commencing with the manufacture of primary containers. The manufacturing process of one embodiment of the invention comprises the following steps:
a) forming (1) of glass primary containers,
b) injection (3) of plastic parts, namely complementary devices (4),
c) assembly (5) of the empty primary containers with corresponding complementary devices to form assembled devices (6),
d) washing (7) the assembled devices with water for injection,
e) nesting (8) the assembled devices in a tray (9),
f) closing a secondary container containing the tray and the assembled devices,
g) sterilising (10) the assembled devices in the closed secondary container,
h) preparing the closed secondary container for transportation to a remote site.

The following operation will be carried at the costumer site:
filling (11) and stoppering the primary containers of the assembled devices,
j) inspecting (12) the assembled devices in a sterile environment,
k) and final packing (13) of the inspected assembled devices.

In contrast, manufacturing processes of the prior art generally comprise the following steps:
a) manufacturing (14) of the primary containers (2),
b) washing (15) the primary containers,
c) nesting (16) the primary containers in a tray,
d) sterilising (17) the primary containers in a secondary container,

The following operation will be carried at the costumer site:
e) filling (18) and stoppering the primary containers,
f) inspecting (19) the primary containers,
g) assembling (20) the primary containers with corresponding complementary devices,
h) inspecting (21) the primary containers assembled with corresponding complementary devices,
i) final packing (22) of the inspected primary containers assembled with corresponding complementary devices.

In the present invention, the primary containers may optionally be syringes or glass containers, pre-fillable with drugs for medical use, and may be formed (1) by a glass-forming device. Any plastic parts, such as complementary devices, may be formed by injection moulding (3). After manufacture, the empty primary containers are assembled (5) with corresponding complementary devices to yield assembled devices (6). In one embodiment of the present invention, the primary containers comprise pre-fillable glass syringes (2). The corresponding complementary devices (4) are safety devices (24) for preventing injury from the bare needle tips of the syringes after injection as well as, optionally, rigid needle shields (25) for preventing injury before injection.

The empty primary containers assembled with the complementary devices must be washed (7), preferably using water for injection. The assembled devices are then transferred into a sterile environment by means of packing them in a secondary container. The secondary container preferably comprises a tray (9) with a plurality of seats into which the assembled devices (6) can be nested such that they are held in a precise position and are held fixedly during transport. This facilitates correct positioning in the x-y plane for accurate filling and stoppering of the devices (6).

The tray (9), which is preferably made of a rigid or semi-rigid plastic material and contains the nested assembled devices (6), is then preferably inserted into a tub of the secondary container. This tub is also preferably made of rigid or semi-rigid plastic and preferably has a transparent section to allow inspection of the contents thereof, in particular the assembled devices. The nest-and-tub design of the secondary packaging is suitable for keeping the assembled devices (6) separate from one another, thereby preventing scratches or other damage.

A peelable sheet of the secondary packaging is applied to the peripheral edge of the tub in order to close the secondary container and seal the assembled devices within the secondary container. The tub is preferably not permeable, whereas the sheet is preferably made of a selectively impervious material that prevents contamination of the assembled devices by micro-organisms, bacteria and/or biologically active materials while remaining permeable to a sterilisation agent such as ethylene oxide gas.

The closed secondary container and its contents are subsequently sterilised (10) and prepared for transfer to a remote site where the remaining steps can be carried out.

After transfer to a different site, the primary containers of the assembled devices (6) are filled (11) with the desired substance, such as drugs for medical use, and stoppered in the sterile environment. In the case where the primary containers are syringes (2), the stoppering comprises insertion of plungers (23) thereinto.

After filling (11) and stoppering, the assembled devices (6) in the sterile environment are visually and/or functionally inspected (12). This can preferably be done either by operators or inspectors and/or by automated systems having one or more cameras and corresponding software. The inspection may check, among other, the colour, amount and other properties of the filled substance, the shape, aesthetics and functional aspects of the primary containers, presence and correct positioning of the stoppers, functionality of moving any parts, and the correct appearance and assembly of the complementary devices (4). The transparent section of the complementary devices (4) allows of the filled primary containers (2) inside the complementary devices (4).

The process may optionally comprise a further step of final packing (13) of the inspected assembled devices (6).

The secondary container and/or the final packaging preferably comprise information for identification and traceability of the contents, such as an RFID system.

The manufacturing process for packing of injectables thus conceived is susceptible to numerous modifications and variations, all falling within the scope of the inventive concept; furthermore, all details may be substituted by technically equivalent elements.

In practice, any material type or size may be used, according to the needs and the state of the art.

1-10. (canceled)

11. A manufacturing process for packing of injectables for medical use, the process comprising in temporal sequence the steps of:
forming of glass primary containers;
forming by plastic injection of complementary devices;
assembling of the empty primary containers with corresponding complementary devices to form assembled devices;
washing the assembled devices with water for injection;
nesting the assembled devices in a tray;
inserting said tray housing said assembled devices into a secondary container and closing said secondary container;
sterilizing the assembled devices in the closed secondary container;
preparing the closed secondary container for transportation to a remote site.

12. The manufacturing process of claim 11, further comprising the step of filling and stoppering said primary containers of said assembled devices by insertion of plungers thereinto.

13. The manufacturing process of claim 12, wherein said tray has a plurality of seats for precise fixation and positioning of said assembled devices in an x-y plane for accurate filling and stoppering thereof.

14. The manufacturing process of claim 11, wherein said primary containers are pre-fillable syringes.

15. The manufacturing process of claim 11, wherein said complementary devices are safety devices for preventing injury from the bare needle tips of said syringes.

16. The manufacturing process of claim 11, wherein said closing step of said secondary container comprises applying a peelable, permeable sheet to the peripheral edge of a tub of said secondary container so as to seal said tray housing said assembled devices within said secondary container.

17. The manufacturing process of claim 11, wherein said assembled devices have at least one transparent section for allowing inspection of said primary filled devices therethrough.

18. The manufacturing process of claim 17, wherein said inspection comprises inspection by an operator.

19. The manufacturing process of claim 17, wherein said inspection comprises automated inspection by at least one camera and corresponding software.

20. The manufacturing process of claim 11, further comprising the step of final packing.
